(19) United States
(12) Patent Application Publication
(10) Pub. No.: US 2025/0186655 A1
(43) Pub. Date: Jun. 12, 2025

(54) VALVE MATERIAL, METHOD FOR PREPARING SAME AND USE THEREOF

(71) Applicant: VENUS MEDTECH (HANGZHOU) INC., Hangzhou (CN)

(72) Inventors: Zhaogang Liu, Hangzhou (CN); Yuting Zhu, Hangzhou (CN); Xing Wei, Hangzhou (CN); Lichao Wang, Hangzhou (CN); Renzheng Ma, Hangzhou (CN)

(21) Appl. No.: 19/057,973

(22) Filed: Feb. 19, 2025

Related U.S. Application Data
(63) Continuation of application No. PCT/CN2023/106865, filed on Jul. 12, 2023.

(30) Foreign Application Priority Data
Aug. 22, 2022 (CN) 202211006505.X

(51) Int. Cl. A61L 27/48; A61F 2/24; A61L 27/50; A61L 27/56

(52) U.S. Cl. CPC A61L 27/48; A61F 2/2415; A61F 2/2418; A61L 27/507; A61L 27/56; A61L 2430/20

(57) ABSTRACT

Disclosed is a polymer valve material, which includes a scaffold, prepared with a first polyurethane as a raw material by electrospinning; and a composite layer, prepared with second polyurethane as a raw material and composited onto the scaffold. The present disclosure further discloses a method for preparing a polymer valve material, including: a spinning step, electrostatic spinning a first polyurethane solution to obtain a prefabricated film; a compositing step, compositing second polyurethane and the prefabricated film to obtain the polymer valve material; and an adjustment step, heat pressing the prefabricated film before or during compositing. The valve material of the present disclosure can improve the creep resistance and edge tear resistance of the valve, as well as extends its service life.

### CROSS-REFERENCE TO RELATED APPLICATIONS

The present application is a Continuation Application of PCT Application No. PCT/CN2023/106865, filed on Jul. 12, 2023, which claims the priority of Chinese Patent Application No. 202211006505.X, filed on Aug. 22, 2022, the entire contents of which are hereby incorporated by reference.

### TECHNICAL FIELD

The present disclosure relates to the technical field of interventional materials, and in particular to a valve material and method for preparing the same and use thereof.

### BACKGROUND

The human heart has four chambers: the left atrium, the left ventricle, the right atrium, and the right ventricle. The two atria communicate with the two ventricles, and the two ventricles communicate with the two aortas. The heart valve is located between the atria and ventricles, as well as between the ventricles and aortas, acting as a one-way valve. The four human valves are the mitral valve, tricuspid valve, aortic valve, and pulmonary valve.

Valvular heart disease is a heart disease caused by stenosis and/or insufficiency of the heart valves due to various reasons. Under normal circumstances, the opening of the heart valve allows blood to flow forward, while the closing of the heart valve prevents blood from flowing back, thereby ensuring one-way flow of blood in the heart. In case of valve stenosis, the pressure load on the heart chamber increases, while in the case of valve insufficiency, the volume load on the heart chamber increases. These hemodynamic changes may lead to structural changes and dysfunction of the atrium or ventricles, ultimately resulting in clinical manifestations such as heart failure and arrhythmias.

Heart Valve Prostheses are prosthetic organs that can be implanted in the heart to replace heart valves (aortic valve, tricuspid valve, mitral valve), allowing one-way blood flow and possessing natural heart valve functions. When the heart valve disease is so severe that the valve function cannot be restored or improved through valve commissurotomy or repair surgery, Prosthetic Heart Valve Replacement is required.

Heart Valve Prostheses are generally categorized into mechanical valves and biological valves, with mechanical valves made entirely of artificial materials and biological valves prepared from biological materials. Pericardial materials are biocompatible and have been used for surgical and interventional valves. However, the source of materials is limited and the materials are poorly homogeneous, requiring a lot of manpower for screening and thus resulting in high costs and low utilization rates. Currently, polymer valves have been widely studied because the valves prepared from polymer materials are easy to process and have a good thickness and performance uniformity. At present, the main polymer materials used for valves include polyurethane and polyetheretherketone. For example, Foldax, RUA Structural Heart, Triskele-UCL, and DSM have disclosed preparation methods for preparing polyurethane materials for medical devices, such as implants, heart valves, and drug delivery devices. These materials have been widely used in the development of implantable medical device products. For example, the literature (Loshini S. Dandeniyage, Development of high strength siloxane poly(urethane-urea) elastomers based on linked macrodiols for heart valve disclosure, Society For Biomaterials, 2017) has shown that the prepared materials have good biocompatibility and are well suited for use as valve materials. However, polymer materials are prone to creep, and the polymer valves are prone to elongation and deformation during use, which affects the fluid performance of the valve. Moreover, polymer materials have poor edge tear strength and are prone to cracking. This is one of the main problems restricting the development of polymer valves.

### SUMMARY

The present disclosure provides a method for preparing a polymer valve material, which improves the creep resistance and edge tear resistance of valves, as well as extends its service life.

A polymer valve material includes:
a scaffold, prepared with a first polyurethane as a raw material by electrospinning; and
a composite layer, prepared with a second polyurethane as a raw material and composited onto the scaffold.

The scaffold has a meshed structure, the second polyurethane fully infiltrates into the scaffold, and finally fills in the meshes of the scaffold and composites onto the surface of the scaffold.

Optionally, the melting point of the first polyurethane is higher than the melting point of the second polyurethane by at least 30° C.

Optionally, the melting point of the first polyurethane is in a range of 180° C. to 300° C.; further, the Shore hardness of the first polyurethane is in a range of 50 D to 90 D, and the number average molecular weight of the first polyurethane is greater than 50,000.

Optionally, the melting point of the second polyurethane is in a range of 100° C. to 240° C.; further, the Shore hardness of the second polyurethane is in a range of 50 A to 80 A, and the number average molecular weight of the second polyurethane is greater than 35,000.

That is, it is preferred that the melting point of the first polyurethane is at least 30° C. higher than that of the second polyurethane; further preferably under this condition, the melting point of the first polyurethane is in a range of 180° C. to 300° C., and the melting point of the second polyurethane is in a range of 100° C. to 240° C.

Optionally, the thickness of the scaffold is in a range of 0.1 mm to 1 mm.

Optionally, the porosity of the scaffold is in a range of 65% to 90%.

Optionally, the thickness of the composite layer is in a range of 0.05 mm to 0.5 mm.

Optionally, the raw materials for electrospinning to prepare the scaffold further include core material, and the core material and the first polyurethane are coaxially electrospun to form the scaffold. That is, in this scheme, the first polyurethane and core material are both used as raw materials to prepare the scaffold by coaxial electrospinning.

The core material should have a strength higher than that of the first polyurethane, to enhance the comprehensive strength of the fiber of the scaffold. The fiber of the scaffold formed by coaxial electrospinning includes a core material disposed on the axis and an outer layer of the first polyurethane wrapped around the core material.

Optionally, in case the first polyurethane complies with the aforementioned conditions, the core material is selected from polyamide (PA) or polyethylene (PE).

A method for preparing a polymer valve material includes:
a spinning step, electrospinning the first polyurethane solution to obtain a prefabricated film;
a compositing step, compositing the second polyurethane with the prefabricated film to obtain the polymer valve material; and

an adjustment step, performing a heat pressing treatment on the prefabricated film before or during the compositing process.

As mentioned above, the selection and relationship between the first polyurethane and the second polyurethane will not be elaborated here.

Optionally, the mass percentage concentration of the first polyurethane solution is in a range of 8% to 18%, and the solvent is a mixed solvent of VTHF/VDMF in a ratio of 1:1 to 5:1, wherein THF is tetrahydrofuran.

Specifically, the high-temperature resistant first polyurethane (with a high content of hard segment) is stirred in the solvent (VTHF/VDMF of 1:1 to 5:1) for 4 to 16 hours to prepare a homogeneous solution with a weight percent of 8% to 18%, which serves as the first polyurethane solution.

Optionally, the first polyurethane may be directly purchased from known commercially available products.

Optionally, the first polyurethane may be prepared from a commonly disclosed polyurethane material synthesis method, such as the synthesis method of polyurethane material used in implantable devices disclosed by Foldax, RUA Structural Heart, Triskele-UCL, DSM. Preferably, the synthesis method described in the literature (Loshini S. Dandeniyage, Development of high strength siloxane poly(urethane-urea) elastomers based on linked macrodiols for heart valve disclosure, Society For Biomaterials, 2017) can be directly used to prepare polyurethane materials with different contents of soft and hard segments.

Optionally, the electrospinning conditions are controlled as follows: a humidity of 45 to 55%, a voltage of 10 to 25 kV, an advancing speed of 0.5 to 1 mL/h, with a spinning distance of 15 to 25 cm, and a needle gauge of 10 G to 30 G.

Optionally, in the spinning step, the first polyurethane solution and the core material solution are coaxially electrospun to obtain a prefabricated film. The interior of the fiber prepared by the preferred scheme of adding the core material is a polymer material with higher strength, which can reduce the stress concentration at the interface between the fiber and the matrix material (i.e., the second polyurethane), and further improve the creep resistance and tear strength of the material; at the same time, the outer layer of the fiber is more compatible with the matrix material.

Optionally, the core material solution is a polyamide (PA) solution or a polyethylene (PE) solution, with a mass percentage concentration of 3% to 10%, and the solvent may be N,N-dimethylformamide (DMF).

In a specific scheme for preparing the PA or PE core material solution: a certain mass of polyamide (with a molecular weight of 15,000 to 30,000) or polyethylene (with a molecular weight of above 1,000,000) is weighed and added in N,N-dimethylformamide (DMF). The mixture is stirred for 4 to 16 hours to dissolve completely, forming a homogeneous solution with a mass concentration of 3% to 10%, which is put aside for later use.

Optionally, the ratio of the core material solution to the first polyurethane solution, calculated based on a mass ratio of the PA or PE core material to the first polyurethane, ranges from 1:5 to 5:1.

Optionally, the method further includes the step of removing residual solvent from the obtained prefabricated film. For example, the prefabricated film may be vacuum-dried at room temperature to remove residual solvent.

Optionally, the thickness of the prefabricated film is in a range of 0.1 to 1 mm.

Optionally, the porosity of the prefabricated film is in a range of 65% to 90%.

Optionally, the prefabricated film is subjected to heat pressing before compositing, and the prefabricated film after heat pressing is dip-coated in a second polyurethane solution or a second polyurethane prepolymer solution.

Optionally, the temperature of the heat pressing treatment is in a range of 100° C. to 240° C., and the time of the heat pressing treatment is in a range of 5 minutes to 2 hours.

Optionally, the thickness of the prefabricated film after heat pressing is in a range of 0.05 to 0.5 mm.

For the scheme of performing dip-coating in the second polyurethane solution:

Optionally, one way to perform dip-coating is to immerse the prefabricated film after the heat pressing treatment in the second polyurethane solution for 4 to 60 seconds and then quickly take it out. This process may be repeated.

Optionally, the mass percentage concentration of the second polyurethane solution is in a range of 20% to 30%.

During the dip-coating process, the prefabricated film prepared from the first polyurethane serves as the scaffold of the material, and the second polyurethane serves as the composite layer of the material, with different requirements for the first polyurethane and the second polyurethane. As a general principle, it is necessary to ensure that the second polyurethane can be well dissolved in the solvent system of the dip-coating step, while the prefabricated film prepared from the first polyurethane still maintains the integrity of the scaffold. For example, DMF or DMAc may be chosen.

Specifically, a certain amount of the second polyurethane is added in DMF or DMAc at the temperature of 60° C. to 90° C. under a nitrogen atmosphere, stirred for 4 to 16 hours, and fully dissolved to obtain a 20 wt % to 30 wt % second polyurethane solution.

Optionally, the second polyurethane may be directly purchased from known commercially available products.

Optionally, the second polyurethane may be prepared by a commonly disclosed polyurethane material synthesis method, such as the synthesis methods of polyurethane materials used in implantable devices disclosed by Foldax, RUA Structural Heart, Triskele-UCL, DSM. Preferably, the synthesis method described in the literature (Loshini S. Dandeniyage, Development of high strength siloxane poly(urethane-urea) elastomers based on linked macrodiols for heart valve disclosure, Society For Biomaterials, 2017) can be directly used to prepare polyurethane materials with different contents of soft and hard segments.

In the present disclosure, there is no clear limit on the number of times the prefabricated film is dip-coated in the second polyurethane solution to achieve the required thickness. Optionally, it generally takes 1 to 3 dips to achieve the required thickness.

For the scheme of performing dip-coating in the second polyurethane prepolymer solution:

Optionally, the synthesis method of the second polyurethane prepolymer solution is as follows: under a nitrogen atmosphere, a pre-polymerization reaction is carried out between the polymer diol and isocyanate; and After the pre-polymerization reaction is complete, the system temperature is lowered to room temperature, and a small-molecule diol chain extender is added and stirred evenly to obtain a second polyurethane prepolymer solution.

Optionally, one implementation method of the dip-coating is immersing the prefabricated film after the heat pressing treatment in the second polyurethane prepolymer solution, and heating it in an oven to perform further chain extension reaction.

Optionally, the heating in the oven is carried out in an anaerobic environment, such as a nitrogen atmosphere, with a heating temperature of 60° C. to 100° C. and a heating time of 3 to 8 h.

Optionally, the heat pressing treatment is performed during the compositing step, wherein the prefabricated film and the second polyurethane particles are placed in a heat pressing mold, and the heat pressing mold is heated to melt the second polyurethane particles while the prefabricated film remains unmelted, thus, the melted second polyurethane fully infiltrates the prefabricated film.

Optionally, the mass ratio of the prefabricated film to the second polyurethane is from 1:3 to 3:1.

The temperature and time of the heat pressing treatment are preferably such that the second polyurethane particles are completely melted while the prefabricated film remains unmelted. Optionally, the temperature of the heat pressing treatment is in a range of 100° C. to 240° C., and the time of the heat pressing treatment is in a range of 5 min to 2 hours. Within this parameter range, the condition of “the second polyurethane particles are completely melted while the prefabricated film remains unmelted” is compliant.

Optionally, the method further includes: performing surface treatment on the polymer valve material obtained in the compositing step. Further, the surface treatment involves performing a plasma treatment on the polymer valve material.

Optionally, the conditions for the plasma treatment are as follows: using SO₂, CO₂, NH₃ or O₂ as the reaction gas, with a power in a range of 100 to 250 W, and a treatment time in a range of 5 min to 15 min.

Specifically, the obtained polymer valve material is placed in a reverse plasma discharge machine with a power in a range of 100 to 250 W, and treated with SO₂, CO₂, NH₃, or O₂ as the reaction gas for 5 to 15 minutes to obtain a plasma treated polymer valve material.

Optionally, the method further includes the step of cutting the plasma treated polymer valve material into leaflet shapes.

The present disclosure further provides a polymer valve material prepared by the above method.

The polymer valve material of the present disclosure may be used for interventional heart valve prosthesis, for example, through minimally invasive intervention; or surgical heart valve prosthesis, for instance, through surgical implantation.

The present disclosure further provides a valve prosthesis, including a stent and a valve material sewn on the stent. The valve material is made of the polymer valve material described in the present disclosure.

Optionally, the valve prosthesis is a heart valve prosthesis.

Compared with the prior art, the present disclosure has at least one of the following beneficial effects:
(1) Good creep resistance;
(2) Strong edge tear resistance; and
(3) Extended service life of the valve.

### BRIEF DESCRIPTION OF DRAWINGS

FIG. 1 is a surface SEM image of an electrospun prefabricated film in Example 1.

FIG. 2 is a surface SEM image of a composite membrane in Example 1.

FIG. 3 is a cross-sectional SEM image of the composite membrane in Example 1.

FIG. 4 is a curve diagram showing elastic deformation changes with the number of cycles for pure polyurethane membrane and the composite membrane in Example 1.

FIG. 5 is a TEM image of a prefabricated film obtained by coaxial electrospinning in Example 3.

FIG. 6 shows a quantitative comparison of platelet adhesion between the surface-modified and non-surface-modified composite membranes in Example 5.

FIG. 7 shows a comparison of contact angles between the surface-modified and non-surface-modified composite membranes in Example 5.

### DESCRIPTION OF EMBODIMENTS

The technical solutions according to the embodiments of the present disclosure will be described clearly and fully in combination with the drawings according to the embodiments of the present disclosure. Apparently, the described embodiments are not all embodiments of the present disclosure, but only part of the embodiments of the present disclosure. Based on the disclosed embodiments, all other embodiments obtained by those skilled in the art without creative work fall into the scope of this invention.

Unless otherwise defined, all technical and scientific terms used herein have the same meaning as commonly understood by a person skilled in the art. The terms in the description of the present disclosure are used to describe specific embodiments, and not to limit the present disclosure.

Polymer materials are prone to creep; thus, the valves prepared from polymer material are prone to elongation and deformation during use, affecting the valve fluid performance. In addition, the edge tear strength of the polymer valve is poor, making it easy to crack, which limits the development of the polymer valve.

In view of the problems of creep and edge tearing in polymer materials, this disclosure proposes adding a nano- or micro-fiber mesh into the polymer material, and then the material is integrally molded to prepare a polymer valve material having the fiber mesh. The purpose is to improve the mechanical properties of the valve by incorporating the fiber mesh, and further enhance the interface bonding strength by infiltration and filling the nano-fiber mesh, thereby improving the creep resistance and tear resistance of the valve.

To solve the above problems, the present disclosure provides, on one aspect, a polymer valve material, which includes a scaffold and a composite layer. The scaffold is a nano- or micro-fiber mesh prepared from a first polyurethane as a raw material by electrospinning; and the composite layer is prepared from a second polyurethane as a raw material and composited onto the scaffold.

The scaffold has a meshed structure, and the second polyurethane fully infiltrates the scaffold, eventually filling the mesh of the scaffold and being composited onto the surface of the scaffold. The thickness of the scaffold prepared by electrospinning may be controlled within a range of 0.1 mm to 1 mm, and the porosity may be controlled within a range of 65% to 90%; the thickness of the composite layer composited onto the scaffold by different methods may be controlled within a range of 0.05 mm to 0.5 mm.

The polymer valve material as mentioned above may be prepared by the improved preparation method of the present disclosure, and the preparation method includes a spinning step, a compositing step, and an adjustment step. In the spinning step, the first polyurethane solution is electrospun to obtain a prefabricated film; in the compositing step, the second polyurethane is composited onto the prefabricated film obtained in the spinning step; and the adjustment step is to perform heat pressing on the obtained prefabricated film before or during the compositing process.

First, a prefabricated film is prepared as a scaffold structure through the spinning step. Then, the second polyurethane is composited onto the scaffold, and the prefabricated film undergoes a heat pressing treatment before or during compositing, to eliminate the obvious interface between the composite layer and the scaffold and improve the bonding strength between the two.

In the present disclosure, polyurethanes with different melting points are selected as materials of the scaffold and the composite layer respectively, and the melting point of the first polyurethane is higher than the melting point of the second polyurethane, so that the prefabricated film prepared from the first polyurethane can maintain the integrity of its scaffold structure when composited with the second polyurethane. Preferably, the melting point of the first polyurethane is higher than that of the second polyurethane by at least 30° C. More preferably, the first polyurethane is selected from a polyurethane having a melting point in a range of 180° C. to 300° C., while the second polyurethane is selected from a polyurethane having a melting point in a range of 100° C. to 240° C.

The first polyurethane that complies with the requirements of the melting point, hardness and molecular weight of this disclosure may be synthesized in-house or purchased as a commercial product that conforms to the corresponding parameters.

The second polyurethane that complies with the requirements of the melting point, hardness and molecular weight of the present disclosure may be synthesized in-house or purchased as a commercial product. Alternatively, the second polyurethane precursor, i.e., the second polyurethane prepolymer, may be synthesized in-house first.

A specific synthesis method of the second polyurethane prepolymer is as follows:
1) Under a nitrogen atmosphere with stirring at 80° C., PTMO (1000 g/mol, 10 g) and PDMS (1000 g/mol, 50 g) (wherein the molar ratio of PTMO to PDMS is 1:5, and total mass is 60 g) are added to MDI (29.375 g) and reacted for 4 h, and 8.02 g of 1,3-bis(4-hydroxybutyl)tetramethyldisiloxane (BHTD) is added dropwise for a pre-polymerization reaction. Then, the system temperature is lowered to room temperature, and 2.59 g of 1,4-butanediol (BDO) (wherein the molar ratio of BHTD to BDO is 1:1) is added and stirred for 30 min; and 2) the prepolymer needs to be kept under a nitrogen atmosphere for 24 hours to obtain a second polyurethane prepolymer solution.

In the electrospinning step, the first polyurethane solution is electrospun to form a prefabricated film. Based on the electrospinning process for preparing the prefabricated film, in the present disclosure, a polymer material with higher strength is further chosen as the internal structure of the fiber, that is, the first polyurethane solution is used as the shell layer solution for coaxial electrospinning with the core material solution. The core material solution is selected from polyamide (PA) or polyethylene (PE) solution with a higher strength.

For the scheme in which the adjustment step is performing a heat pressing treatment before compositing, the prefabricated film obtained by spinning is firstly subjected to heat pressing treatment, and then the prefabricated film after heat pressing treatment is dip-coated in the second polyurethane solution or a second polyurethane prepolymer solution. In this scheme, the temperature of heat pressing treatment is preferably in a range of 125° C. to 180° C., and the time of heat pressing treatment is preferably in a range of 5 min to 2 h.

In one embodiment of performing the dip-coating treatment in the second polyurethane solution, a certain amount of the second polyurethane is added in DMF or DMAc under a nitrogen atmosphere at a temperature of 60 to 90° C., stirred for 4 to 16 hours, and fully dissolved to obtain a 20 wt % to 30 wt % second polyurethane solution; and the prefabricated film after the heat pressing treatment is immersed in the second polyurethane solution for 4 to 60 seconds and then quickly taken out. This process may be repeated.

In one embodiment of performing the dip-coating treatment in the second polyurethane prepolymer solution, the second polyurethane prepolymer solution should be prepared first. The prefabricated film after the heat pressing treatment is immersed in the second polyurethane prepolymer solution, and then taken out and heated in an oven for further chain extension reaction.

In the scheme of combining heat pressing before compositing and performing dip-coating in the second polyurethane prepolymer solution, heat pressing before compositing can enhance the strength of the prefabricated film, but at the same time, it will also respond to the compression of the pores of the prefabricated film. However, when dip-coating in the second polyurethane in the form of a prepolymer, due to its smaller molecular weight and shorter chain segments compared with high molecular weight polyurethane, the prepolymer is more likely to enter the scaffold formed by the first polyurethane during the dip-coating process, and the system has better fusion and more complete molecular motion. Then, the chain extension polymerization is further achieved during the drying process. On the one hand, the scheme of performing dip-coating in prepolymer can lengthen the chain length of the second polyurethane. On the other hand, the polymerization of part of the polyurethane occurs between molecules inside the electrospun fiber, thus further eliminating the interface, improving compatibility, and enhancing the overall strength while also ensuring better interface bonding of the composite membrane (i.e., the polymer valve material).

For the scheme in which the adjustment step is performing a heat pressing treatment during compositing, the prefabricated film obtained in the spinning step and the second polyurethane particles are placed in a heat pressing mold, and the heat pressing mold is heated to melt the second polyurethane particles while the prefabricated film remains unmelted. Thus, the melted second polyurethane fully infiltrates into the prefabricated film, and then the prefabricated film is heat pressed to obtain a composite membrane, which is used as the polymer valve material. The temperature and time of the heat pressing treatment are preferably such that the second polyurethane particles are completely melted while the prefabricated film remains unmelted. In an optional scheme, the temperature of heat pressing treatment is in a range of 100° C. to 240° C., and the time of heat pressing treatment is in a range of 5 min to 2 h.

In the scheme of heat pressing during the compositing process, the prefabricated film is relatively thicker and has larger pores, the second polyurethane is easier to infiltrate, and the interface bonding of the composite membrane is better. At the same time, heat pressing during the compositing process can also improve the overall strength of the composite membrane; thus, the composite membrane prepared by this scheme has a better strength and interface bonding.

In order to further improve the biocompatibility of the polymer valve material, in the present disclosure, a surface treatment is further performed on the composite membrane. In a preferred surface treatment method, plasma technology is used to perform surface treatment on the polymer valve material. In a specific implementation method, the obtained polymer valve material is placed in an inverse plasma discharge machine with a power of 100 to 250 W, and treated with SO₂, CO₂, NH₃, or O₂ as the reaction gas for 5 to 15 minutes to obtain a plasma treated polymer valve material. Experiments have shown that the surface-modified polymer valve material has good anti-platelet adhesion.

The following is described with specific examples:

### Example 1

(1) A first polyurethane with high-temperature resistance (melting point: 195° C., Shore hardness: 80 D) was stirred in a solvent (VTHF/VDMF=1:1) for 4 h to prepare a homogeneous solution with a weight percent of 10%, serving as the first polyurethane solution.

Under a nitrogen atmosphere with stirring at 80° C., PTMO (1000 g/mol, 10 g) was added to MDI (9.97 g) and reacted for 2 h, and 1.35 g of 1,4-butanediol (BDO) was added dropwise for chain extension reaction. After reacting for 2 h, the system temperature was lowered to room temperature, and 0.90 g of ethylenediamine (EDA) (wherein the molar ratio of BDO to EDA was 1:1) was added dropwise. The reaction was carried out for 4 h to prepare the first polyurethane solution.

The obtained first polyurethane solution was electrospun under the following conditions: a humidity of 50%, a voltage of 15 kV, an advancing speed of 1 mL/h, a spinning distance of 20 cm and a needle gauge of 21 G to obtain a prefabricated film with a thickness of 0.8 to 1 mm.
(3) The prefabricated film was vacuum dried at room temperature for 48 h to remove residual solvent.
(4) The prefabricated film after removing the residual solvent was subjected to heat pressing treatment, with a heat pressing temperature of 170° C. and a heat pressing time of 10 minutes. The thickness of the prefabricated film after heat pressing treatment is in a range of 0.08 to 0.12 mm.
(5) A certain amount of a second polyurethane (melting point: 160° C., Shore hardness: 80 A) was taken and completely added in DMF under nitrogen atmosphere at 80° C., which was then stirred for 4 h to obtain a 10 wt % second polyurethane solution.

Under a nitrogen atmosphere with stirring at 80° C., PTMO (1000 g/mol, 10 g) and PDMS (1000 g/mol, 50 g) (wherein the mass ratio of PTMO to PDMS was 1:5, and total mass was 60 g) were added to MDI (29.375 g) to react for 4 h, and 8.02 g of 1,3-bis(4-hydroxybutyl)tetramethyldisiloxane (BHTD) was added dropwise for chain extension reaction. After reacting for 2 h, 2.59 g of 1,4-butanediol (BDO) (wherein the molar weight of BHTD to BDO was 1:1) was added to react for 4 h to obtain the second polyurethane solution.
(6) The prefabricated film after the heat pressing treatment in step (4) was immersed in the second polyurethane solution prepared in step (5) and quickly taken out (with an immersion time of 4 to 6 seconds). After that, it was dried in a nitrogen atmosphere at 60° C. to 80° C. for 24 h to obtain a polymer valve material (i.e., a composite membrane) with a reinforced meshed structure inside, and the valve thickness was 0.15 mm.

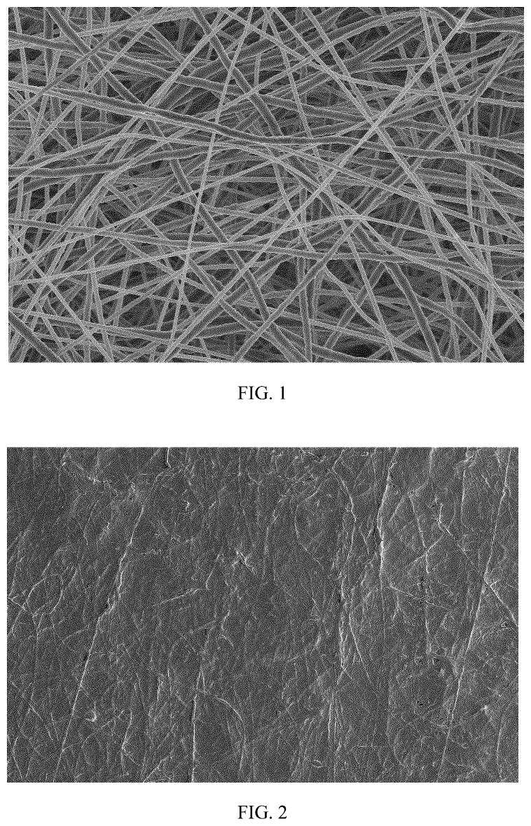

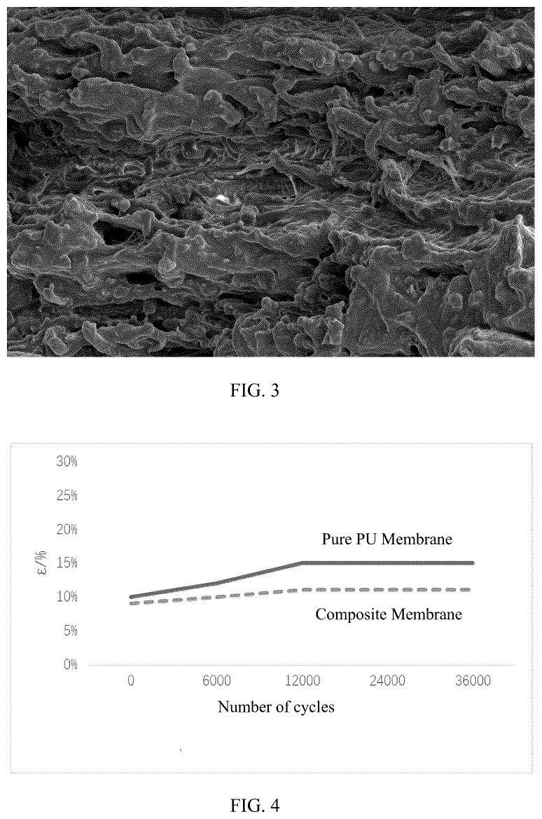

The surface SEM image of the electrospun prefabricated film material prepared in step (3) of Example 1 is shown in FIG. 1, and the surface SEM image and cross-sectional SEM image of the composite membrane in step (6) are shown in FIG. 2 and FIG. 3. As can be seen from the results in FIGS. 2 to 3, the fiber mesh is wrapped by the second polyurethane solution without obvious interface stratification, indicating that this step improves the bonding strength between the fiber mesh and the polyurethane material.

The mechanical properties of a pure polyurethane membrane (formed by conventional methods such as pouring the second polyurethane solution into a mold with a flat surface and waiting for the solvent to evaporate) and the composite membrane were measured according to standards of ASTM D412 and ASTM D 624. The test results are shown in Table 1.

TABLE 1

| | | Mechanical | Elongation | Tear |
|---|---|---|---|---|
| Sample | Thickness | strength | at break | resistance |
| Pure polyurethane | 0.15 to | 26 Mpa | 680% | 50 N/min |
| membrane | 0.18 mm | | | |
| Composite | 0.15 mm | 30 Mpa | 300% | 75 N/min |
| membrane of | | | | |
| Example 1 | | | | |

Compared with the pure polyurethane membrane, the composite membrane prepared by compositing the electrospun fiber mesh with the polyurethane material helps to improve the mechanical strength and edge tear resistance of the valve. At the same time, heat pressing can control the thickness of the electrospun valve.

A single load test was carried out to test the creep performance of the material, wherein a creep test was conducted at room temperature with an applied stress of 2 MPa. The frequency was set to 1 Hz, and a total of 36,000 cycles were performed to test the elastic deformation under different numbers of cycles. The test results are shown in FIG. 4. Compared with the pure polyurethane membrane, the composite membrane shows stable elastic deformation at 10% after 36000 cycles, indicating its excellent creep resistance.

### Example 2

(1) A first polyurethane with high-temperature resistance (melting point: 195° C., Shore hardness: 80 D, which were the same as that in Example 1) was stirred in a solution (VTHF/VDMF=1:1) for 4 h to prepare a homogeneous solution with a weight percent of 10%, serving as the first polyurethane solution.
(2) The obtained first polyurethane solution was electrospun under the conditions of a humidity of 50%, a voltage of 15 kV, an advancing speed of 1 mL/h, a spinning distance of 20 cm and a needle gauge of 21 G to obtain a prefabricated film with a thickness of 0.8 to 1 mm.
(3) The prefabricated film was vacuum dried at room temperature for 48 h to remove residual solvent.
(4) The prefabricated film after removing the residual solvent was subjected to heat pressing treatment, with a heat pressing temperature of 170° C. and a heat pressing time of 10 minutes. The thickness of the prefabricated film after heat pressing treatment is 0.10 mm.
(5) Preparation of second polyurethane prepolymer solution.
1) Under a nitrogen atmosphere with stirring at 80° C., PTMO (1000 g/mol, 10 g) and PDMS (1000 g/mol, 50 g) (wherein the mass ratio of PTMO to PDMS was 1:5, and total mass was 60 g) were added dropwise to MDI (29.375 g) to react for 4 h, and 8.02 g of 1,3-bis(4-hydroxybutyl)tetramethyldisiloxane (BHTD) was added dropwise for chain extension reaction. Then, the system temperature was lowered to room temperature, and 2.59 g of 1,4-butanediol (BDO) (wherein the mass ratio of BHTD to BDO was 1:1) was added and stirred for 30 min.
2) The prepolymer was kept under a nitrogen atmosphere for 24 hours to obtain a second polyurethane prepolymer solution.
(6) The prefabricated film after the heat pressing treatment in step (4) was immersed in the second polyurethane solution prepared in step (5) and quickly taken out (with an immersion time of 4 to 6 seconds). It was then reacted in an oven at 80° C. under a nitrogen atmosphere for 4 h, and subsequently dried at 60° C. for 24 h. Thus, a composite membrane with a thickness of 0.15 mm was obtained.

The mechanical properties of a pure polyurethane membrane (formed by conventional methods such as pouring the second polyurethane solution into a mold with a flat surface and waiting for the solvent to evaporate) and the composite membrane were measured according to standards of ASTM D412 and ASTM D 624. The test results are shown in Table 2.

TABLE 2

| | | Mechanical | Elongation | Tear |
|---|---|---|---|---|
| Sample | Thickness | strength | at break | resistance |
| Pure polyurethane | 0.15 to | 26 Mpa | 680% | 50 N/min |
| membrane | 0.18 mm | | | |
| Composite | 0.15 mm | 32 Mpa | 320% | 78 N/min |
| membrane | | | | |
| of Example 2 | | | | |

### Example 3

(1) A first polyurethane with high-temperature resistance (melting point: 195° C., and Shore hardness: 80 D, which were the same as those in Example 1) was stirred in a solution (VTHF/VDMF=1:1) for 4 h to prepare a homogeneous solution with a weight percent of 10%, which was set aside as the shell layer solution.
(2) A certain amount of high molecular weight polyethylene (molecular weight: 1.5 million) was weighed and added into N,N-dimethylformamide (DMF) solution. It was stirred for 8 hours to be completely dissolved, and a homogeneous solution with a mass percentage concentration of 10% was obtained, which was reserved as the core material solution.
(3) The core material solution and the shell layer solution (the mass ratio of polyethylene to the first polyurethane calculated based on the mass ratio of the core material solution and the shell layer solution was 1:1) were coaxially electrospun to obtain a prefabricated film with a thickness in a range of 0.8 to 1 mm. The electrospinning parameters were as follows: a humidity of 50%, a voltage of 15 kV, an advancing speed of 1 mL/h, with a spinning distance of 25 cm, and a needle gauge of 17 G to 22 G.
(4) The prefabricated film was vacuum dried at room temperature for 48 h to remove residual solvent.
(5) The prefabricated film treated in step (4) was placed in a heat pressing mold, second polyurethane particles (melting point: 160° C., Shore hardness: 80 A, which were the same as those in Example 1, and the mass ratio of the prefabricated film to the second polyurethane particles was 1:1) were added into the heat pressing mold, which was then heated at a temperature of 170° C. for a heating time of 10 min, so that the second polyurethane particles were completely melted and the prefabricated film remained unmelted.
(6) After being fully infiltrated by the melted second polyurethane, the prefabricated film was integrally mold-pressed to form a polymer valve material with a reinforced mesh structure in the middle part, and the valve thickness was 0.15 mm. The fiber that constitutes the mesh structure has a polymer material filament structure with higher strength inside.

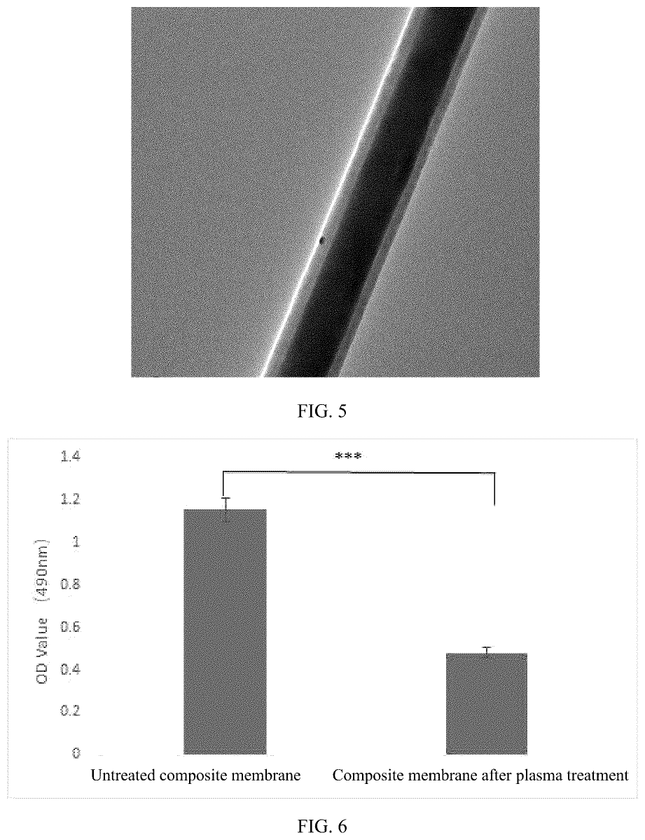

TEM image of the prefabricated film prepared by coaxially electrospun in step (4) in Example 3 is shown in FIG. 5.

The mechanical properties of a pure polyurethane membrane (formed by conventional methods such as pouring second polyurethane solution into a mold with a flat surface and waiting for the solvent to evaporate) and the composite membrane were measured according to standards of ASTM D412 and ASTM D 624. The test results are shown in Table 3.

TABLE 3

| | Mechanical | Elongation | |
|---|---|---|---|
| Sample | strength | at break | Tear resistance |
| Pure polyurethane | 26 Mpa | 680% | 50 N/min |
| membrane | | | |
| Composite membrane | 35 Mpa | 280% | 80 N/min |
| of Example 3 | | | |

Compared with the pure polyurethane membrane and the fiber mesh formed by electrospinning, a polymer polyethylene material with higher strength is chosen as the inner layer of the fiber mesh prepared by coaxial electrospinning, which further enhances the mechanical strength of the composite membrane. At the same time, a polyurethane material that is more compatible with the matrix is chosen as the outer layer, which results in better compatibility between the fibers and the matrix material and reduced stress concentration at the interface. The test results indicate that the composite membrane prepared by coaxial electrospinning has a stronger mechanical strength and edge tear resistance.

### Example 4

(1) A first polyurethane with high-temperature resistance (melting point: 195° C., Shore hardness: 80 D, which were the same as those in Example 1) was stirred in a solution (VTHF/VDMF=1:1) for 4 h to prepare a homogeneous solution with a weight percent of 10%, serving as the first polyurethane solution.

The obtained first polyurethane solution was electrospun under the conditions of humidity of 50%, voltage of 15 kV, advancing speed of 1 mL/h, spinning distance of 20 cm and needle gauge of 21 G to obtain a prefabricated film with a thickness of 0.8 to 1 mm.
(3) The prefabricated film was vacuum dried at room temperature for 48 h to remove residual solvent.
(4) The prefabricated film treated in step (3) was placed in a heat pressing mold, second polyurethane particles (melting point: 160° C., Shore hardness: 80 A, which were the same as those in Example 1, and the mass ratio of the prefabricated film to the second polyurethane particles was 1:1) were added into the heat pressing mold, which was then heated at a temperature of 170° C. for a heating time of 10 min, so that the second polyurethane particles were completely melted and the prefabricated film remained unmelted, and the melted second polyurethane fully infiltrated the prefabricated film.
(5) The infiltrated prefabricated film was integrally mold-pressed to form a polymer valve material with a reinforced mesh structure in the middle. The valve thickness was in a range of 0.15 to 0.20 mm.

The mechanical properties of a pure polyurethane membrane (formed by conventional methods such as pouring the second polyurethane solution into a mold with a flat surface and waiting for the solvent to evaporate) and the composite membrane were measured according to standards of ASTM D412 and ASTM D 624. The test results are shown in Table 4.

TABLE 4

| | Mechanical | Elongation | |
|---|---|---|---|
| Sample | strength | at break | Tear resistance |
| Pure polyurethane | 26 Mpa | 680% | 50 N/min |
| membrane | | | |
| Composite membrane of | 30 Mpa | 300% | 75 N/min |
| Example 4 | | | |

### Example 5

(1) A first polyurethane with high-temperature resistance (melting point: 195° C., and Shore hardness: 80 D, which were the same as those in Example 1) was stirred in a solution (VTHF/VDMF=1:1) for 4 h to prepare a homogeneous solution with a weight percent of 10%, which was set aside as the shell layer solution.

The obtained shell layer solution was electrospun under the following conditions: a humidity of 50%, a voltage of 15 kV, an advancing speed of 1 mL/h, a spinning distance of 20 cm and a needle gauge of 21 G, to obtain a prefabricated film with a thickness of 0.8 to 1 mm.
(3) The prefabricated film was vacuum dried at room temperature for 48 h to remove residual solvent.
(4) The prefabricated film treated in step (3) was placed in a heat pressing mold, the second polyurethane particles (melting point: 160° C., Shore hardness: 80 A, which were the same as those in Example 1, and the mass ratio of the prefabricated film to the second polyurethane particles was 1:1) were added into the heat pressing mold, which was then heated at a temperature of 170° C. for a heating time of 10 min, so that the second polyurethane particles were completely melted and the prefabricated film remained unmelted, and the melted second polyurethane fully infiltrated the prefabricated film.
(5) The infiltrated prefabricated film was integrally mold-pressed to form a polymer valve material (i.e., the composite membrane) with a reinforced mesh structure in the middle. The valve thickness was in a range of 0.15 to 0.20 mm.
(6) The composite membrane obtained in step (5) was placed in a plasma discharge machine with a power of 200 W, and treated with SO₂ as the reaction gas for 10 min to obtain a plasma-treated polymer valve material.

The LDH content released by the platelets adsorbed on the material was measured to test the adhesion of the material to platelets.

Specific experimental processes were as follows:
(1) After the sample membrane was exposed to fresh platelet-rich plasma at 37° C. for 30 minutes, the membrane was carefully washed with PBS; (2) After natural drying in the air, 150 μL of LDH release reagent diluted 10 times with PBS was added to the 96-well plate, which was shaken to mix evenly, and then placed in a 37° C. incubator to incubate for 1 hour; (3) 120 μL of supernatant from each well was taken and added to a new 96-well plate, and then three solutions (lactic acid solution, enzyme solution and 1×INT solution) were added in a volume ratio of 1:1:1 to prepare the detection solution. 60 μL of detection solution was added to the supernatant of each well, mixed, and incubated on a horizontal shaker for 20 to 30 minutes under light-proof conditions at room temperature, and then the absorbance of the solution at 490 nm was detected using an enzyme reader. The test results are shown in FIG. 6, indicating that the surface-treated polymer valve material has good anti-platelet adhesion.

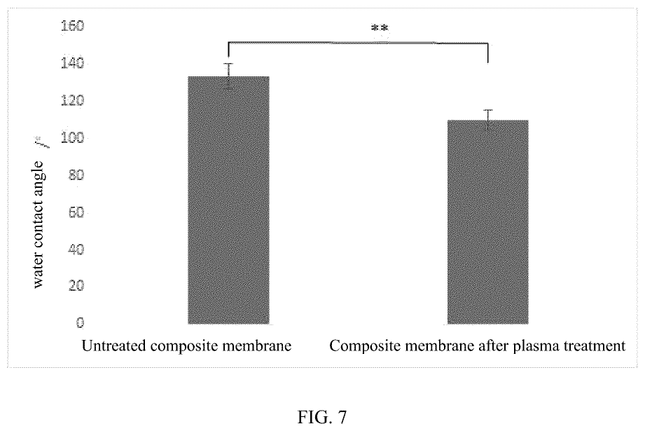

A SZ10-JC2000C contact angle meter was used to measure the contact angle between water and the sample. The test results are shown in FIG. 7, indicating that the surface-treated polymer valve material has a small contact angle and good hydrophilicity.

The above-described embodiments only illustrate several embodiments of the present disclosure, and the description thereof is specific and detailed, but should not be construed as limiting the scope of the patent disclosure. It should be noted that, for those of ordinary skill in the art, several modifications and improvements can be made without departing from the concept of the present disclosure, all of which fall into the protection scope of the present disclosure. Therefore, the protection scope of the present disclosure should be determined by the appended claims.

What is claimed is:

1. A polymer valve material, comprising:
a scaffold, prepared with a first polyurethane as a raw material by electrospinning; and
a composite layer, prepared with a second polyurethane as a raw material and composited onto the scaffold;
wherein a melting point of the first polyurethane is higher than a melting point of the second polyurethane with a difference therebetween of at least 30° C.

2. The polymer valve material according to claim 1, wherein the melting point of the first polyurethane is in a range of 180° C. to 300° C., and a Shore hardness of the first polyurethane is in a range of 50 D to 90 D.

3. The polymer valve material according to claim 1, wherein the melting point of the second polyurethane is in a range of 100° C. to 240° C., and a Shore hardness of the second polyurethane is in a range of 50 A to 80 A.

4. The polymer valve material according to claim 1, wherein a thickness of the scaffold is in a range of 0.1 mm to 1 mm, a porosity of the scaffold is in a range of 65% to 90%, and a thickness of the composite layer is in a range of 0.05 mm to 0.5 mm.

5. The polymer valve material according to claim 1, wherein the raw material for preparing the scaffold by electrospinning further comprises a core material, which is coaxially electrospun to form the scaffold with the first polyurethane, wherein the first polyurethane is wrapped around the core material, and the core material has a strength higher than a strength of the first polyurethane.

6. The polymer valve material according to claim 5, wherein the core material is polyamide (PA) or polyethylene (PE).

7. A method for preparing a polymer valve material, comprising:
a spinning step, electrospinning a first polyurethane solution to obtain a prefabricated film;
a compositing step, compositing a second polyurethane with the prefabricated film to obtain the polymer valve material; and
an adjustment step, performing a heat pressing treatment on the prefabricated film before or during the compositing process.

8. The method for preparing a polymer valve material according to claim 7, wherein in the spinning step, the first polyurethane solution and a core material solution are coaxially electrospun to obtain the prefabricated film; and
the core material solution is a polyamide (PA) solution or a polyethylene (PE) solution.

9. The method for preparing a polymer valve material according to claim 8, wherein a ratio of the core material solution to the first polyurethane solution, calculated based on a mass ratio of the PA or PE core material to the first polyurethane, ranges from 1:5 to 5:1.

10. The method for preparing a polymer valve material according to claim 8, wherein a thickness of the prefabricated film is in a range of 0.1 mm to 1 mm, and a porosity of the prefabricated film is in a range of 65% to 90%.

11. The method for preparing a polymer valve material according to claim 8, wherein the adjustment step is performing a heat pressing treatment on the prefabricated film before the compositing step, wherein after the heat pressing treatment, the prefabricated film is dip-coated in a second polyurethane solution or a second polyurethane prepolymer solution for compositing, and during dip-coating, a solvent of the second polyurethane solution or the second polyurethane prepolymer solution is DMF or DMAc.

12. The method for preparing a polymer valve material according to claim 11, wherein during dip-coating, the prefabricated film after the heat pressing treatment is immersed in the second polyurethane solution for 4 seconds to 60 seconds and then taken out, and a mass percentage concentration of the second polyurethane solution is in a range of 20% to 30%.

13. The method for preparing a polymer valve material according to claim 11, wherein when preparing the second polyurethane prepolymer solution, polymer diol and isocyanate are first subjected to a pre-polymerization reaction; and
after the pre-polymerization reaction is completed, a small-molecule diol chain extender is added for reaction to obtain a second polyurethane prepolymer solution.

14. The method for preparing a polymer valve material according to claim 11, wherein during dip-coating, when the prefabricated film after the heat pressing treatment is immersed in the second polyurethane prepolymer solution, a heating condition is further required to carry out a chain extension reaction.

15. The method for preparing a polymer valve material according to claim 8, wherein the adjustment step is performing heat pressing treatment during the compositing process, which comprises placing the prefabricated film and second polyurethane particles in a heat pressing mold, and heating the heat pressing mold to melt the second polyurethane particles while the prefabricated film remains unmelted, such that melted second polyurethane infiltrates the prefabricated film.

16. The method for preparing a polymer valve material according to claim 14, wherein a mass ratio of the prefabricated film to the second polyurethane is in a range of 1:3 to 3:1, the heat pressing treatment is performed at a temperature in a range of 100° C. to 240° C., and time of the heat pressing treatment is in a range of 5 min to 2 h.

17. The method for preparing a polymer valve material according to claim 8, further comprising performing a plasma treatment on the polymer valve material obtained in the compositing step, and wherein conditions for the plasma treatment are as follows: using SO₂, CO₂, NH₃ or O₂ as reaction gas, with a power in a range of 100 to 250 W, and a treatment time in a range of 5 min to 15 min.

18. A polymer valve material prepared by the preparation method according to claim 7.

19. A heart valve prosthesis, comprising a stent and a valve material sewn on the stent, wherein the valve material is the polymer valve material according to claim 18.

20. A heart valve prosthesis, comprising a stent and a valve material sewn on the stent, wherein the valve material is the polymer valve material according to claim 1.
